(19) United States
(12) Patent Application Publication
(10) Pub. No.: US 2006/0153932 A1
(43) Pub. Date: Jul. 13, 2006

(54) Method of treating hyperphosphataemia using lanthanum hydroxycarbonate

(75) Inventors: Josephine C. Ferdinando, Hampshire (GB); David Gilmour, Middlesex (GB)

Correspondence Address: DARBY & DARBY P.C., P. O. BOX 5257, NEW YORK, NY, 10150-5257, US

(73) Assignee: Shire Pharmaceuticals, Inc.

(21) Appl. No.: 11/191,600

(22) Filed: Jul. 27, 2005

Related U.S. Application Data
(60) Provisional application No. 60/591,105, filed on Jul. 27, 2004.

(51) Int. Cl. A61K 33/24

(52) U.S. Cl. 424/617

(57) ABSTRACT

This disclosure relates to the treatment of subjects at risk for chronic kidney disease (CKD), having stage one to five CKD, having hyperphosphataemia, susceptible to or suffering from soft tissue calcification associated with CKD, or susceptible to or suffering from hyperparathyroidism, by orally administering a pharmaceutical composition containing a therapeutically effective amount of lanthanum hydroxycarbonate.

This application claims priority to U.S. Provisional Application Ser. No. 60/591,105 filed on Jul. 27, 2004.

### FIELD OF THE INVENTION

This invention relates to the treatment of subjects at risk for chronic kidney disease (CKD), having stage one to five CKD, having hyperphosphataemia, susceptible to or suffering from soft tissue calcification associated with CKD, or susceptible to or suffering from hyperparathyroidism, by orally administering a pharmaceutical composition containing a therapeutically effective amount of lanthanum hydroxycarbonate (La(OH)CO₃).

### BACKGROUND OF THE INVENTION

Chronic kidney disease (CKD) is a worldwide public health problem. According to a National Health and Nutrition Examination Survey (NHANES), the number of CKD subjects in the United States will increase from approximately 26 million in 2004 to approximately 40 million in 2020. One of the major complications of CKD is elevated blood phosphate levels resulting from the inability of the kidney to remove phosphate from the body by urine secretion. Excess phosphate levels in the blood result in CKD subjects developing hyperphosphataemia. The number of CKD subjects with hyperphosphataemia in the United States will increase from approximately 1 million in 2005 to approximately 2.8 million in 2020.

Hyperphosphataemia is a particular problem for patients with chronic renal insufficiency who are using dialysis equipment and for about 70% of patients with end stage renal disease (ESRD). This condition can lead to severe bone problems and metastatic calcification of major organs and is associated with significant morbidity and mortality. Conventional dialysis fails to reduce the levels of phosphate in the blood, so that levels rise in time. Elevated phosphate levels are treated using a combination of dietary restrictions and phosphate-binding agents.

Currently, the Food and Drug Administration (FDA) has limited the treatment of hyperphosphataemia using phosphate binders to subjects with “End-Stage Renal Disease” (ESRD), i.e., stage five of CKD. This sub-population of CKD subjects represents only 1% of the total CKD subject population.

Hyperphosphataemia in ESRD subjects can be controlled using calcium-based phosphate binders, sevelamer (i.e., a positively-charged polymer available, e.g., as Renagel® Tablets (sevelamer hydrochloride) from Genzyme in Cambridge, Mass.), and aluminum-based binders. Subjects who receive calcium-based binders often are unable to achieve desired phosphate levels without exceeding their recommended daily intake of calcium and are burdened with the amount of drug they must take. Additionally, calcium-based binders may cause hypercalcaemia and exacerbate ectopic calcification as described, infra. Subjects who are prescribed sevelamer also have an unmanageably large pill burden due to the lack of potency of this drug. Aluminum-based binders, although highly potent and efficacious, are associated with central nervous system and bone toxicity when used over long periods.

Another problem of patients with chronic renal insufficiency is secondary hyperparathyroidism. It is also important in patients with chronic renal insufficiency to avoid and treat secondary hyperparathyroidism.

Certain forms of lanthanum carbonate have been used to treat hyperphosphataemia in patients with renal failure (see, e.g., JP 1876384). U.S. Pat. No. 5,968,976 assigned to Shire Pharmaceuticals discloses a pharmaceutical composition comprising a lanthanum carbonate hydrate having the formula La₂(CO₃)₃.xH₂O, where x has a value between 3 to 6, to treat hyperphosphataemia in ESRD subjects. Processes for preparing this composition and a method to treat hyperphosphataemia in ESRD subjects using this composition are also described.

Lanthanum carbonate tetrahydrate in the form of a chewable tablet (available as Fosrenol® from Shire Pharmaceuticals, Wayne, Pa.) has also been approved by the FDA to treat hyperphosphatemia in ESRD subjects. Unlike other problematic phosphate binders, lanthanum carbonate-based binders are potent with a manageable dosing regimen, do not cause hypercalcemia, and are non-toxic over long periods.

Patent applications WO 02/085348 and US 2002/155168 (Use of rare earth compounds for the prevention of kidney stone diseases; Abrams et al) relate to a method of preventing or treating urolithiasis (kidney stone disease) by administering rare earth salts, e.g., lanthanum salts, to bind dietary oxalate and preventing its absorption into the gastrointestinal tract.

Patent application US 2002/0051822 relate to the administration of a lanthanum compound for enhancing bone formation, inhibiting osteoclastic differentiation and/or activating osteoclastic differentiation thereby managing, treating or achieving prophylaxis of bone disease.

### SUMMARY OF THE INVENTION

There exists a need for an agent, which can be used to treat the above conditions in patients suffering from a variety of clinical disorders, e.g., in renal failure patients or patients with a bone disorder, wherein, e.g., the level of phosphate in the serum of the patient can be maintained at homeostasis levels with preventing, reducing, or abolishing incidences of hyperphosphataemia.

This invention relates to a method of treating a subject (1) at risk for CKD, (2) having stage one to stage five CKD, or (3) susceptible to or suffering from soft tissue calcification associated with CKD, comprising orally administering a pharmaceutical composition containing as an active ingredient a therapeutically effective amount of lanthanum hydroxycarbonate (La(OH)CO₃).

This invention also relates to a method for controlling or treating hyperphosphataemia in a patient comprising administering a therapeutically effective amount of lanthanum hydroxycarbonate.

The invention further provides a pharmaceutical composition comprising said lanthanum hydroxycarbonate, in admixture or association with a pharmaceutically acceptable diluent or carrier, in a form for administration into the gastrointestinal tract for the treatment of hyperphosphataemia.

The invention may also be expressed as a method of treatment of hyperphosphataemia in a patient with renal failure, comprising the administration of an effective dose of said lanthanum hydroxycarbonate into the gastrointestinal tract.

This invention relates to a method for controlling or treating hyperphosphataemia in a patient comprising administering a therapeutically effective amount of lanthanum hydroxycarbonate in a formulation which achieves desirably low plasma levels of lanthanum.

The invention may also be expressed as a method for treating hyperparathyroidism in a patient with chronic renal insufficiency comprising administering a therapeutically effective amount of lanthanum hydroxycarbonate.

### BRIEF DESCRIPTION OF THE DRAWINGS

FIG. 1 compares the vitro phosphorus binding ability of lanthanum hydroxycarbonate tetrahydrate with that of lanthanum carbonate.

### DETAILED DESCRIPTION OF THE INVENTION

In accordance with the present invention, a method of treating a subject (1) at risk for CKD, (2) having stage one to stage five CKD, (3) susceptible to or suffering from soft tissue calcification associated with CKD, or (4) susceptible to or suffering from hyperparathyroidism is provided, comprising orally administering a pharmaceutical composition containing as an active ingredient a therapeutically effective amount of lanthanum hydroxycarbonate (La(OH)CO₃). As indicated hereinafter, the invention is applicable to the treatment of subjects exhibiting one or more functional or structural abnormalities indicating risk for, susceptibility to, or informing the diagnosis of any of stages one to five of CKD, soft tissue calcification associated with such CKD, or hyperparathyroidism. When lanthanum hydroxycarbonate is administered to such a subject, it is possible to reduce if not arrest the progress of CKD, soft tissue calcification associated with CKD, and/or hyperparathyroidism.

In one embodiment, the invention relates to such a method for treating hyperphosphataemia in a renal failure patient, including but not limited to a patient receiving dialysis and/or a patient with end-stage renal disease (ESRD), comprising administering a therapeutically effective amount of lanthanum hydroxycarbonate.

In another embodiment, the formulation for the lanthanum compound comprises lanthanum hydroxycarbonate, diluent, blending/flow agents and lubricants.

In another embodiment, the formulation for the lanthanum compound comprises various tablet dosages as shown in the table below:

| Formulation | % range by wt. |
|---|---|
| Lanthanum hydroxycarbonate | 20-60% |
| Diluent | 40-80% |
| (e.g., dextrates (hydrated)) | |
| Blending/flow agents & lubricants | 0.01-10% |
| (e.g., colloidal anhydrous silica, | |
| magnesium stearate) | |

In another embodiment, this invention relates to treating a patient with hyperparathyroidism or with hypercalcaemia (e.g., underlying calcium based treatment for hyperphosphataemia, supra) by administering lanthanum hydroxycarbonate.

In another embodiment, lanthanum hydroxycarbonate is administered such that plasma levels of lanthanum are low, at least as low as those obtained from the administration of lanthanum carbonate tetrahydrate. Lanthanum needs to be locally available in the gastrointestinal tract where it can effectively bind phosphate. e.g., plasma levels at least as low as those provided by a concentration curve where Cₘₐₓ, Tₘₐₓ and AUC are preferably less than 1.5 ng/ml, about 12 hours, and less than 50 ng·hr/ml, respectively, for a dose of 3 g/day (e.g., 1 g three times a day), such as is achieved in prior art formulations of lanthanum carbonate tetrahydrate. In a more preferred embodiment, Cₘₐₓ and AUC are less than 1.1 ng/ml and less than 32 ng·hr/ml at such dosage, and in a most preferred embodiment, Cₘₐₓ and AUC are less than 0.5 ng/ml and less than 20 ng·hr/ml at such dose. Tₘₐₓ values are essentially unaffected by dose and Cₘₐₓ and AUC values vary linearly with dosage.

The term “AUC” as used herein, means area under the plasma concentration-time curve, as calculated by the trapezoidal rule over the complete dosing interval, e.g., 24-hour interval.

The term “Cₘₐₓ” as it is used herein is the highest plasma concentration of the drug attained within the dosing interval.

The term “tₘₐₓ” as it is used herein is the time period which elapses after administration of the dosage form at which the plasma concentration of the drug attains the Cₘₐₓ within the dosing interval.

By “pharmaceutically acceptable,” such as in the recitation of a “pharmaceutically acceptable carrier,” is meant herein a material that is not biologically or otherwise undesirable, i.e., the material may be incorporated in a pharmaceutical composition administered to a patient without causing any undesirable biological effects or interacting in a deleterious manner with any of the other components of the composition in which it is contained.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

The terms “effective amount” and “therapeutically effective amount” of a drug or pharmacologically active agent are synonymous and refer to a nontoxic but sufficient amount of the drug or agent to provide the desired effect. In the combination therapy of the present invention, an “effective amount” of one component of the combination is the amount of that component that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual. In the context of the present invention, treatment refers particularly to the treatment of hyperphosphataemia or hyperparathyroidism with administration of an effective amount of lanthanum hydroxycarbonate. For example, treatment of a subject at risk for or having one of stages one to five of CKD can mean the reduction of abnormally high serum phosphate levels; the prevention of soft tissue calcification; or the reduction of abnormally elevated parathyroid hormone (PTH) levels.

The term “combination therapy”, in defining use of lanthanum hydroxycarbonate with one or more additional pharmaceutical agents, is intended to embrace administration of each agent in a sequential manner in a regimen that will provide beneficial effects of the drug combination, and is also intended to embrace co-administration of the pharmaceutical agents in a substantially simultaneous manner, such as in a single capsule having a fixed ratio of active ingredients (i.e., a unit dose) or in multiple, separate capsules for each pharmaceutical agent.

The term “lanthanum hydroxycarbonate” is used herein to denote any pharmacologically acceptable lanthanum hydroxycarbonate compound capable of binding phosphate.

The term “symptom(s)” of those at risk for or having CKD, soft tissue calcification associated with CKD, or secondary hyperparathyroidism may be any functional or structural abnormality experienced by a subject and indicating kidney dysfunction as described herein. For example, one or more of the following symptoms may indicate risk for or the presence of CKD: a creatinine concentration of above about 1.6 mg/dL, a blood urea nitrogen (BUN) of above about 20 mg/dL, a blood phosphate level of above about 4.5 mg/dL, any detectable amount of blood in the urine, a urine protein concentration above about 100 mg/dL, a urine albumin concentration above about 100 mg/dL, an intact parathyroid hormone (PTH) concentration in the blood of above about 150 pg/mL, or a glomerular filtration rate (GFR) of below about 90 mL/min/1.73 m².

The National Kidney Foundation-Kidney Disease Outcomes Quality Initiative (“NKF-K/DOQI” or “K/DOQI,” as referred to herein) has defined chronic kidney disease (CKD) as either (1) having kidney damage as defined by structural or functional abnormalities of the kidney for 3 months or longer with or without a decreased glomerular filtration rate (GFR) or (2) having a GFR of less than 60 mL/min/1.73 m² for 3 months or longer with or without kidney damage.

Examples of markers of kidney damage include a plasma creatinine concentration of above about 1.6 mg/dL and a blood urea nitrogen (BUN) concentration of above about 20 mg/dL. Typically, both of these markers are elevated in individuals with CKD. Additional markers of kidney damage can include hematuria (i.e., any detectable amount of blood in the urine) proteinuria (i.e., protein concentrations in urine above about 100 mg/dL), albuminuria (i.e., albumin concentrations in urine above about 100 mg/dL), an intact parathyroid hormone (PTH) concentration in the blood above about 150 pg/mL, or blood phosphate levels of above about 4.5 mg/dL. One specific marker of kidney disease is a GFR rate above normal (i.e., a GFR above about 90 mL/min/1.73 m²), however a below normal GFR also indicates CKD.

K/DOQI has published guidelines that define five different stages of CKD (Am J Kidney Dis. 2001, 37(suppl 1):S1-S238). The following table provides a description of each of the five stages of CKD and the GFR ranges for each of the stages, as well as the GFR rates characterizing subjects at risk of CKD.

Five Stages of Chronic Kidney Disease (CKD)

| | | GFR |
|---|---|---|
| Stage | Description | (mL/min/1.73 m²) |
| | At risk | 90-120 (with |
| | | CKD symptoms) |
| 1 | Kidney damage with normal or elevated GFR | ≧90 |
| 2 | Kidney damage with mildly reduced GFR | 60-89 |
| 3 | Moderately reduced GFR | 30-59 |
| 4 | Severely reduced GFR | 15-29 |
| 5 | Kidney Failure (ESRD) | <15 (or dialysis) |

Hyperphosphataemia in CKD subjects has several secondary effects. When a subject suffers from hyperphosphataemia, excess serum phosphate precipitates serum calcium causing widespread ectopic extraskeletal calcification. Unwanted calcium deposits can occur in cardiovascular tissue, resulting in an increased risk of cardiovascular complications that often lead to death. Additionally, increased serum phosphate decreases intestinal calcium absorption. These two mechanisms work concurrently to reduce serum calcium levels.

A reduction in serum calcium levels can contribute to an increase in the production of parathyroid hormone (PTH) and to the development of secondary hyperparathyroidism. Furthermore, recent studies show that high phosphate levels can stimulate PTH production directly and lead to secondary hyperparathyroidism. Continual stimulation of PTH secretion induces hyperplasia of the parathyroid gland and may lead to a parathyroidectomy becoming necessary.

It is believed that the method of the present invention involving the administration of lanthanum hydroxycarbonate not only reduces plasma phosphate levels but ameliorates the effects of CKD in subjects susceptible to or having any of stages one to five CKD, including hyperphosphataemia, ESRD, ectopic extraskeletal calcification, serum hypocalcaemia, and secondary hyperparathyroidism. It should however, be understood that this invention is not limited to any particular biochemical or physiological mechanism.

One embodiment of this invention is a method of treating a subject having a symptom or symptoms of chronic kidney disease (CKD), comprising administering to the subject a pharmaceutical composition containing as an active ingredient a therapeutically effective amount of a lanthanum hydroxycarbonate. As indicated above, the subject treated may be at risk for CKD or have any of stages one to five CKD as defined above. Subjects at risk for CKD or who have any of stages one to five CKD who may be treated may have one or more of the following symptoms: a blood phosphate level of above about 4.5 mg/dL, a plasma creatinine concentration of above about 1.6 mg/dL, a BUN of above about 20 mg/dL, any detectable amount of blood in the urine, a urine protein concentration above about 100 mg/dL, a urine albumin concentration above about 100 mg/dL, an intact parathyroid hormone concentration in the blood above about 150 pg/mL, an abnormal GFR, or combination thereof.

The present method may be utilized to prevent the progression of renal pathology, e.g., by treating a subject displaying one or more symptoms of stage one CKD to prevent the development of CKD in the subject or by treating a subject having stage one CKD to prevent progression of the disease to stage two CKD, and so on.

The inventors have found that, surprisingly, lanthanum hydroxycarbonate has improved rates of in vitro phosphate binding as compared to lanthanum carbonate tetrahydrate when dosed at the same level of elemental lanthanum. Lanthanum hydroxycarbonate is approximately 50% more effective at binding phosphate in vitro at pH 1. Even at the same level of elemental lanthanum, the lanthanum hydroxycarbonate has a lower molecular weight, 18.5% lower, than lanthanum carbonate tetrahydrate, i.e., one has to dose approximately 1.2 g of lanthanum carbonate tetrahydrate to get the same level of elemental lanthanum as only 1 g of lanthanum hydroxycarbonate due to the molecular weight difference. Both these aspects of improved phosphate binding and lower molecular weight allows a reduction in tablet size of approximately 20-50%, when using lanthanum hydroxycarbonate compared to lanthanum carbonate tetrahydrate. This is because not only is the weight of the active ingredient reduced due to the lower molecular weight and potent binding efficiency in relation to the active lanthanum element, but also the excipient weights can be reduced since there is less active ingredient present. The reduced tablet size is a great benefit to the convenience of the patient and could also allow the production of even higher dose tablets. Increasing the dose of elemental lanthanum per tablet also allows reduction in the number of tablets taken, which again is a benefit to the patient. Additionally the lower molecular weight of lanthanum hydroxycarbonate compared to lanthanum carbonate tetrahydrate means that smaller tablets can be made to achieve the same dose. Smaller tablets have the benefit of significantly reduced tablet chipping potential. By necessity, chewable tablets are made softer than conventional swallowable tablets, this makes them vulnerable if they are large and heavy to chipping as a consequence of tablets hitting each other or hard surfaces during manufacture or transit. Smaller, lighter tablets have a much reduced tendency to chip, this means the quality of tablet appearance is improved.

Lanthanum hydroxycarbonate also has no associated water hydration and so does not need controlled and lengthy drying. Lanthanum carbonate tetrahydrate is made from lanthanum carbonate octahydrate which must be dried in a controlled manner for many hours to achieve the tetrahydrate status. Lanthanum hydroxycarbonate has no required hydration status and is therefore more easily and rapidly manufactured.

Lanthanum hydroxycarbonate may be synthesized by methods known in to those skilled in the art including, (1) from hydrated lanthanum(III) carbonate under hydrothermal conditions as disclosed in Haschke, J., Journal of Solid State Chemistry, 12 (1975) 115-121; (2) from LaBr(OH)₂ treated with carbon dioxide or from hydrolysis of lanthanum carbonate as disclosed in Sun, J.; Kyotani, T.; Tomita, A. J. Solid State Chem., 65 (1986) 94; (3) the treatment of lanthanum(III) nitrate with urea or thiourea as disclosed in Han et al. Inorganic Chemistry Communications, 6 (2003) 117-1121; (4) the treatment of lanthanum(III) chloride with urea or thiourea as disclosed in Han et al. Journal of Solid State Chemistry, 177 (2004) 3709-3714; (5) the treatment of lanthanum(III) chloride with trifluoroacetic acid as disclosed in Wakita, H et al., Bulletin of the Chemical Society of Japan, 52 (1979) 428-432; or (6) the treatment of lanthanum(III) chloride with sodium carbonate as disclosed in Nagashima, K et al. Bulletin of the Chemical Society of Japan, 46 (1973) 152-156.

A study was conducted to assess the toxicity of lanthanum hydroxycarbonate. Rats were dosed orally with lanthanum hydroxycarbonate (103 or 1030 mg lanthanum/kg/day), lanthanum carbonate tetrahydrate (103 or 1030 mg lanthanum/kg/day) or vehicle for 4 weeks.

Plasma exposure to lanthanum was similar in the groups receiving the hydroxycarbonate and carbonate salts. This study indicated that the hydroxycarbonate salt of lanthanum has a very similar toxicity profile to the carbonate salt.

In a further embodiment, the invention is directed to a method to remove oxalate in a subject which method comprises administering to the gastrointestinal tract of said subject, an effective amount of lanthanum hydroxycarbonate.

In a further embodiment, the invention is directed to a method to inhibit the formation of kidney stones in a subject which method comprises administering to the gastrointestinal tract of said subject, an effective amount of lanthanum hydroxycarbonate.

When the compositions of the invention are used for removal of oxalates from the gastrointestinal tract; administration of these compositions is preferably to the upper digestive tract, most conveniently by oral administration. The compounds are effective over a pH range encountered in these locations which ranges from pH 1 in the stomach to pH 8 in regions downstream thereof. The compositions of the invention are not subject to degradation at high pH, and thus it is unnecessary to take special precautions, such as the supply of enteric coatings for oral administration.

The conditions characterized by kidney stones are believed to be related to inappropriate absorption of oxalate from the intestinal tract; inhibition of such absorption appears useful in controlling this condition. While not intending to be bound by any theory, applicants specifically include kidney stones among conditions that are affected by excessive oxalate absorption from the gastrointestinal tract. In addition, inappropriate absorption of oxalate from the gastrointestinal tract is itself a condition which requires remediation. The sequelae of such inappropriate absorption include the symptomology of kidney stones, but other deposits of oxalate may form in other organs as well or the levels of oxalate in the bloodstream may themselves be deleterious. Thus, any subject who exhibits levels of oxalate in the blood or serum that are higher than a normal level is also a candidate for treatment according to the method of the invention. Methods for determining oxalate levels in the diet and in the bloodstream or serum are known in the art.

Lanthanum hydroxycarbonate can be formulated and used in essentially the same manner as the other lanthanum compounds. In an advantageous aspect, oral tablets of a given unit dosage can be smaller and lighter than for lanthanum compounds containing water of hydration, e.g., by a factor of 1.5-3.5, or lower or higher values.

Lanthanum is a rare earth element with an atomic number of 57. The properties of lanthanum make this agent a good candidate as a useful phosphate binder. It has a high affinity for binding phosphorous. In addition, the phosphate binding is independent of pH, it possesses a low toxic potential based on the LD₅₀, it is palatable, abundant, and has limited effects on serum electrolyte concentrations (Hutchison, A J et al. (1998) Perit. Dial. Int. 18(Suppl 2): S38).

The lanthanum compound of the invention may be administered in the form of a pharmaceutical composition comprising the active ingredient in admixture or association with a pharmaceutically acceptable carrier or excipients. The active ingredient may be formulated into a composition suitable for administration by any convenient route, oral administration being preferred. It should be understood, however, that the invention embraces all pharmaceutically acceptable forms of administration which make the lanthanum locally available.

Orally administrable compositions may, if desired, contain one or more physiologically compatible carriers and/or excipients and may be solid or liquid. The compositions may take any convenient form including, for example, tablets, coated tablets, capsules, lozenges, suspensions, emulsions, syrups, elixirs and dry products suitable for reconstitution with water or another suitable liquid vehicle before use. The compositions may advantageously be prepared in dosage unit form. Tablets and capsules according to the invention may, if desired, contain conventional ingredients such as binding agents, for example syrup, acacia, gelatin, dextrates, sorbitol, tragacanth or polyvinyl-pyrollidone; fillers/diluents, for example lactose, sugar, maize-starch, calcium phosphate, sorbitol or glycine; lubricants and/or flow aids, for example magnesium stearate, purified talc, polyethylene glycol or silica (e.g., colloidal anhydrous silica); disintegrants, for example potato starch; or acceptable wetting agents such as sodium lauryl sulfate. Tablets may be coated according to methods well known in the art. In a preferred embodiment, lanthanum hydroxycarbonate is administered orally in a tablet. In a further embodiment, the tablet is a chewable tablet. Excipients and processes for preparing formulations are well known in the art, for example see Lieberman et al., Pharmaceutical Dosage Forms, Marcel Dekker, Inc, New York, e Ed. Vol 1-3 (1990).

Liquid compositions may contain conventional additives such as suspending agents, for example sorbitol syrup, methyl cellulose, glucose/sugar syrup, gelatin, hydroxymethylcellulose, carboxymethylcellulose, aluminium stearate gel or hydrogenated edible fats; emulsifying agents, for example lecithin, sorbitan monooleate or acacia; non-aqueous vehicles, which may include edible oils, for example vegetable oils such as arachis oil, almond oil, fractionated coconut oil, medium chain triglycerides, fish-liver oils, oily esters such as polysorbate 80, propylene glycol, or ethyl alcohol; and preservatives, for example methyl or propyl p-hydroxybenzoates or sorbic acid. Liquid compositions may conveniently be encapsulated in, for example, gelatin to give a product in dosage unit form.

It may be advantageous to incorporate an antioxidant, for example ascorbic acid, butylated hydroxyanisole or hydroquinone in the compositions of the invention to enhance their storage life.

It will be understood that the dosages of compositions and the duration of administration according to the invention will vary depending on the requirements of the particular subject. The precise dosage regime will be determined by the attending physician or veterinary surgeon who will, inter alia, consider factors such as body weight, age and symptoms (if any). The compositions may if desired incorporate one or more further active ingredients.

During the dosing regimen, administration may be effected one or more times per day, for example once, twice, three or four times per day.

The invention further provides a pharmaceutical composition comprising the lanthanum hydroxycarbonate, in admixture or association with a pharmaceutically acceptable diluent or carrier, in a form for administration for the treatment of hypercalcaemia.

With respect to therapeutic agents, it is expected that the skilled practitioner will adjust dosages on a case by case basis using methods well established in clinical medicine. Nevertheless, the following general guidelines with respect to lanthanum hydroxycarbonate may be of help

Without limiting the scope of the present invention, a typical dosage of lanthanum hydroxycarbonate for an adult may be, e.g., from about 715 to about 8586 mg daily which is equivalent to from about 375 to about 4500 mg elemental lanthanum. The dose can be divided and taken with meals, for example from about 125 to about 1500 mg elemental lanthanum/meal (e.g., three times per day). Serum plasma levels can be monitored weekly until an optimal serum phosphate level is reached conventionally. Administration may be conducted in an uninterrupted regimen; such a regimen may be a long term regimen, e.g., a permanent regimen, for treating chronic conditions.

Lanthanum hydroxycarbonate can be administered in tandem with other drugs which are used to treat a variety of clinical disorders including but not limited to cardiovascular ailments. The lanthanum hydroxycarbonate compound can be administered once per day for several consecutive days followed by administration of the other drug. Also, the other drug, as for example digoxin, warfarin or metoprolol, can be administered first followed by lanthanum hydroxycarbonate. Also, the other administered agent can be administered using any regimen which is conventionally used for the agent. If two or more active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts.

The dosage regimens set forth herein are simply guidelines since the actual dose must be carefully selected and titrated by the attending physician based upon clinical factors unique to each patient. The optimal daily dose will be determined by methods known in the art and will be influenced by factors such as the age of the patient, the disease state, side effects associated with the particular agent being administered and other clinically relevant factors. In some cases, a patient may already be taking medications at the time that treatment is initiated. These other medications may be continued provided that no unacceptable adverse side effects are reported by the patient.

Often, a subject suffering from the symptoms of CKD is also vitamin D deficient because, his or her kidney can no longer metabolize vitamin D prohormones into the active metabolite of vitamin D; and increased phosphate levels found in CKD subjects are believed to suppress the production of the active metabolite of vitamin D. In another embodiment of the present invention, the lanthanum hydroxycarbonate, in combination with vitamin D or an analog of vitamin D, is administered to a subject suffering from the symptoms of CKD to alleviate vitamin D deficiency.

Examples of vitamin D sources which may be so administered concurrently with the lanthanum hydroxycarbonate in this invention include 1,25 dihydroxy-vitamin D, the active metabolite of vitamin D (calcitriol, rocalcitrol). Examples of suitable vitamin D analogs include doxercalciferol (Hectorol®, available from Bone Care International, Middleton, Wis.), paricalcitol (Zemplar®, available from Abbott Laboratories, Abbott Park, Ill.),

Vitamin D can be formulated and administered using routes as described, supra. Vitamin D can be combined in the same formulation as the lanthanum hydroxycarbonate or can be given in a different formulation as the lanthanum hydroxycarbonate. As described above for lanthanum hydroxycarbonate, the precise dosage regimen for vitamin D will be determined by the attending physician or veterinarian who will, inter alia, consider factors such as body weight, age and specific symptoms. The physician or veterinarian may titrate the dosage of vitamin D administered to a subject to determine the correct dosage for treatment.

In a specific embodiment, 100 USP units of vitamin D is administered once per day and lanthanum hydroxycarbonate is administered three times per day to a subject requiring treatment.

As described above, CKD subjects often suffer from hypocalcaemia (i.e., a blood calcium concentration below about 8.5 mg/dL). In a further embodiment of this invention, lanthanum hydroxycarbonate is administered in combination with a calcium source to a subject suffering from the symptoms of CKD. It is noted that some patients with hyperphosphataemia may be suffering from hypercalcaemia due to a prior administration of a calcium-based treatment. Therefore the administration of a calcium source with lanthanum hydroxycarbonate should be carefully considered based on the patient's blood calcium concentration.

Examples of forms of calcium that can be co-administered with lanthanum hydroxycarbonate include calcium carbonate (e.g., Tums® available from GlaxoSmithKline, Uxbridge, UK), calcium acetate (e.g., PhosLo® available from Nabi Biopharmaceuticals, Boca Raton, Fla.), and CaCl₂.

Calcium dosages (expressed as elemental calcium) can range from 1 to 1.5 grams/day. A calcium compound can be combined in the same formulation with the lanthanum hydroxycarbonate or can be given in a different formulation as the lanthanum hydroxycarbonate. A calcium compound, whether in the presence or absence of lanthanum hydroxycarbonate in the same formulation, can be formulated and administered using routes as described, supra. The exact dosage regimen for calcium will be determined by the attending physician or veterinarian who will, inter alia, consider factors such as body weight, age and specific symptoms. The physician or veterinarian may titrate the dosage of calcium administered to a subject to determine the correct dosage for treatment.

In a specific embodiment, 1-2 tablets containing calcium and lanthanum hydroxycarbonate are each given 3 times per day.

Any of the routes and regimens of administration may be modified depending on any superior or unexpected results which may be obtained as routinely determined with this invention.

Without further elaboration, it is believed that one skilled in the art can, using the preceding description, utilize the present invention to its fullest extent. The following examples are, therefore, to be construed as merely illustrative, and not limiting the scope of the invention in any way whatsoever.

### EXAMPLE 1

### Evaluation of In vitro Phosphorus Binding of Lanthanum Compounds

In order to evaluate the efficacy of lanthanum hydroxycarbonate as a phosphate binder, the in vitro phosphorus binding of lanthanum hydroxycarbonate and lanthanum carbonate tetrahydrate were measured using the methodology described below:

The equivalent of 1 g elemental lanthanum as either lanthanum hydroxycarbonate or as lanthanum carbonate tetrahydrate was added to 500 mL of 0.1N HCl at 37° C. and adjusted to pH 1 with HCl which contained 300 mg of phosphorus. The preparation was stirred and sampled at regular intervals. This sample was filtered and the filtrate measured for phosphorus content using a Sigma Diagnostics Kit for Inorganic Phosphorus determination. The loss of phosphorus from the filtrate represents the amount of phosphorus bound by lanthanum and precipitated then retained on the filter.

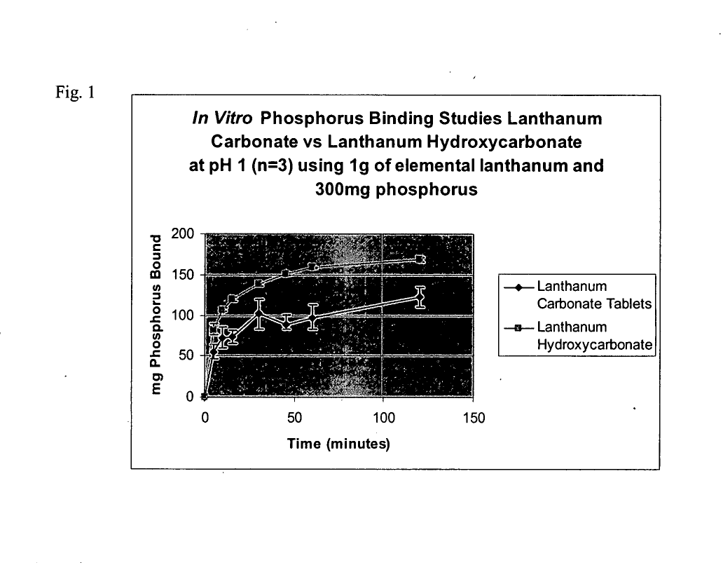

The results are presented on FIG. 1 and show the phosphate binding capability of lanthanum hydroxycarbonate compared to lanthanum carbonate tetrahydrate.

The preceding examples can be repeated with similar success by substituting the generically or specifically described reactants and/or operating conditions of the invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

What is claimed is:

1. A method of treating a subject (1) at risk for chronic kidney disease (CKD), (2) having stage one to stage five CKD, or (3) susceptible to or suffering from soft tissue calcification associated with CKD, comprising orally administering to the subject a pharmaceutical composition containing as an active ingredient a therapeutically effective amount of lanthanum hydroxycarbonate.

2. The method of claim 1, wherein the subject is suffering from hyperphosphataemia.

3. The method of claim 1, wherein the lanthanum hydroxycarbonate is administered to the subject in a daily dose ranging from 715 to 8586 mg.

4. A method of claim 1, wherein the subject is suffering from renal failure, receiving dialysis or has end-stage renal disease.

5. A method of treating hyperparathyroidism comprising administering to a patient in need thereof a therapeutically effective amount of lanthanum hydroxycarbonate.

6. The method of claim 5, wherein the lanthanum hydroxycarbonate is administered to the subject in a daily dose ranging from 715 to 8586 mg.

7. A method of claim 5, wherein said patient is suffering from renal failure, receiving dialysis or has end-stage renal disease.

8. A pharmaceutical composition comprising lanthanum hydroxycarbonate and a pharmaceutically acceptable carrier.

9. A pharmaceutical composition of claim 8, wherein the lanthanum hydroxycarbonate is present in from about 20 to about 60 percent by weight.
